(19) United States
(12) Patent Application Publication
(10) Pub. No.: US 2022/0096514 A1
(43) Pub. Date: Mar. 31, 2022

(54) METHODS OF TREATING THYMIDINE KINASE 2 DEFICIENCY BY ADMINISTERING DEOXYCYTIDINE AND DEOXYTHYMIDINE

(71) Applicant: ZOGENIX INTERNATIONAL LIMITED, Berkshire (GB)

(72) Inventor: Joanne QUAN, Emeryville, CA (US)

(73) Assignee: ZOGENIX INTERNATIONAL LIMITED, Berkshire (GB)

(21) Appl. No.: 17/388,378

(22) Filed: Jul. 29, 2021

Related U.S. Application Data
(60) Provisional application No. 63/084,954, filed on Sep. 29, 2020.

(51) Int. Cl. A61K 31/7072; A61K 31/7068; A61P 21/00

(52) U.S. Cl. CPC A61K 31/7072; A61P 21/00; A61K 31/7068

(57) ABSTRACT

Provided is a method of treating thymidine kinase 2 (TK2) deficiency in a subject by administering deoxycytidine (dC) and deoxythymidine (dT). The method can include administering dC and dT to a subject in doses between 200 mg/kg/day and 600 mg/kg/day for each of dC and dT. In some cases, the method includes performing test administrations with lower dosages of dC and dT, administering adjusted doses after administration of the target doses, or a combination thereof.

### INTRODUCTION

Thymidine kinase 2 (TK2) deficiency is a rare and commonly lethal disorder caused by autosomal recessive or de novo mutations in the TK2 gene resulting in mitochondrial DNA (mtDNA) depletion, multiple deletions, or a combination thereof. This enzyme localizes to the mitochondria of a cell. The TK2 enzyme is directly responsible for phosphorylation of pyrimidine nucleosides needed to replicate mtDNA and its functional impairment results in decreased mitochondrial DNA copy number in affected tissues. Patients can have different symptoms, and hence TK2 deficiency can be considered a clinically heterogeneous disorder. In most or all patients progressive muscle weakness is observed, as a high requirement for mitochondrial DNA encoded proteins in muscle predispose this tissue to the devastating effect of TK2 deficiency. Other symptoms involving motor function deficiencies, problems with breathing, need for a feeding tube, impaired growth, neurologic deficits and death. TK2 deficiency is a type of mitochondrial DNA depletion syndrome (MDDS). The TK2 gene encodes for a deoxyribonucleoside kinase that phosphorylates thymidine, deoxycytidine, and deoxyuridine.

### SUMMARY

Provided is a method of treating thymidine kinase 2 (TK2) deficiency in a subject by administering deoxycytidine (dC) and deoxythymidine (dT). The method can include administering dC and dT to a subject in doses between 200 mg/kg/day and 600 mg/kg/day for each of dC and dT. In some cases, the method includes performing initial administrations with lower dosages of dC and dT, administering adjusted doses after administration of the target doses, or combinations thereof.

### BRIEF DESCRIPTION OF THE DRAWINGS

FIG. 1 provides a generalized method of treatment. The solid box surrounding “target doses” indicates that administering target doses of deoxycytidine (dC) and deoxythymidine (dT) is performed. The dashed boxes surrounding the other three steps indicate that those steps are optional.

FIG. 2 consist of three parts with the top flow chart showing an exemplary method wherein all four steps are performed, as indicated by the four solid boxes surrounding each of the four steps. The graph in the middle represents the administration of dC and graph at the bottom represents the administration of dT with both being administered in equal doses. The doses of dC and dT administered change depending on which step of the method in the flow chart at the top currently being performed.

FIG. 3 consists of 3A, 3B, 3C, 3D, and 3F which schematically represent possible options for performing the generalized method of FIG. 1. In particular: in FIG. 3A only the target administration is performed; in FIG. 3B the target and adjusted administrations are performed; in FIG. 3C the second and target administrations; in FIG. 3D the second, target, and adjusted administrations; in FIG. 3E the first, second, and target administrations; in FIG. 3F all four of the steps.

FIG. 4 shows an overview of a study design.

FIG. 5 shows dose titration and reduction schedules for the study design of FIG. 4.

FIG. 6 shows the indications of treatment efficacy that were used to monitor each subject depending on the age of the subject.

FIG. 7 shows a detailed discussion of the types of motor function assessments employed during the monitoring.

### DETAILED DESCRIPTION

Provided is a method of treating thymidine kinase 2 (TK2) deficiency in a subject by administering deoxycytidine (dC) and deoxythymidine (dT). The method can include administering dC and dT to a subject in doses between 200 mg/kg/day and 600 mg/kg/day for each of dC and dT. In some cases, the method includes performing initial administrations with lower dosages of dC and dT, administering adjusted doses after administration of the target doses, or combinations thereof. Any age of patient may be treated according to the methods provided herein.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a droplet” includes a plurality of such droplets and reference to “the discrete entity” includes reference to one or more discrete entities, and so forth. It is further noted that the claims may be drafted to exclude any element, e.g., any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

Deoxycytidine is sometimes abbreviated herein as “dC”. As used herein, deoxycytidine and dC refer not only to the free base form of the compound, but also salts thereof. The free base of deoxycytidine has the Chemical Abstract Services (CAS) number of 951-77-9 and the chemical structure shown below.

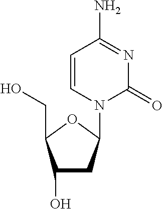 [SMILES: Nc1ccn([C@H]2C[C@@H](O)[C@@H](CO)O2)c(=O)n1]

Deoxythymidine is sometimes abbreviated herein as “dT” and is also sometimes referred to as “thymidine”. As used herein, deoxythymidine and dT refer not only to the free base form of the compound, but also salts thereof. The free base of deoxythymidine has the CAS number of 50-89-5 and the chemical structure shown below.

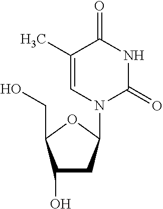 [SMILES: Cc1cn([C@H]2C[C@@H](O)[C@@H](CO)O2)c(=O)[nH]c1=O]

When referring to administration of a compound to a subject, the terms “dose”, “amount”, and “dosage” are used interchangeably. The units of mg/kg/day refers to how many milligrams of a compound is administered to the subject within a 24 hour period per kilogram of body mass of the patient. For example, a patient weighing 100 kg and receiving 200 mg/kg/day of deoxycytidine would receive 20,000 mg of deoxycytidine within a 24 hour period. The units mg/kg/day can also be written as mg/(kg·day) or mg·kg⁻¹·day⁻¹.

The term “MT1621” refers to a pharmaceutical composition including equal doses of dC and dT.

“Administering target doses” can also be referred to as “target doses administration” or “target administration”. Similarly, administering first doses can also be referred to as the first administration, and administering second doses can also be referred to as the second administration. Administering adjusted doses can be alternatively referred to as the adjusted administration.

The terms active agent, active pharmaceutical ingredient, pharmacologically active agent, and drug are used interchangeably herein to refer to a chemical material or compound which, when administered to an organism (human or animal) induces a desired pharmacologic and/or physiologic effect by local and/or systemic action.

The terms “individual,” “host,” “subject,” and “patient” are used interchangeably herein, and refer to a human of any age, including “infants” (birth through 2 years), “children” (from 3 up to 10 years), “adolescents” (10 to 19 years) and “adults” (20 years and older). Non-human animal models, e.g., mammals, e.g. non-human primates, murines, lagomorpha, etc. may be used for experimental and non-clinical investigations.

As used herein, the terms “treatment,” “treating,” and the like, refer to obtaining a desired pharmacologic and/or physiologic effect, such as reduction of viral titer. The effect may be prophylactic in terms of completely or partially preventing a disease or symptom thereof and/or may be therapeutic in terms of a partial or complete cure for a disease and/or adverse symptom attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease (as in liver fibrosis that can result in the context of chronic HCV infection); (b) inhibiting the disease, i.e., arresting its development; and (c) relieving the disease, i.e., causing regression of the disease (e.g., reduction in viral titers or improvement in disease biomarkers). The age at onset of symptoms, rate of weakness progression and post-onset survival are defining variables of three clinical subtypes of TK2 deficiency. The subtypes are classified as infantile-onset myopathy (birth to two years of age), childhood-onset myopathy (between 3 years and 12 years) and late on-set disease. Nervous system involvement often complicates the clinical course of the infantile-onset form which typically progresses faster to disability and death while extraocular muscle and facial involvement are characteristic of the late-onset form along with a longer post-onset survival time.

A “therapeutically effective dose”, a “therapeutically effective dose” or “therapeutic dose” is an dose sufficient to effect desired clinical results (i.e., achieve therapeutic efficacy, achieve a desired therapeutic response, etc.). A therapeutically effective dose can be administered in one or more administrations. For purposes of this disclosure, a therapeutically effective dose of a compositions is an dose that is sufficient, when administered to the individual, to palliate, ameliorate, stabilize, reverse, prevent, slow or delay the progression of a disease state (e.g., cancer, etc.) present in the subject.

As used herein, the terms “monitoring,” “determining,” “measuring,” “assessing,” and “assaying” are used interchangeably and include both quantitative and qualitative determinations.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

### Methods

Provided are methods of treating thymidine kinase 2 (TK2) deficiency in a subject with deoxycytidine (dC) and deoxythymidine (dT). Such methods include administering target doses of dC and dT to the subject, wherein the target doses are each independently between 200 mg/kg/day and 600 mg/kg/day. In some cases, the method also includes administering first doses of dC and dT, administering second doses of dC and dT, administering adjusted doses of dC and dT, or a combination thereof.

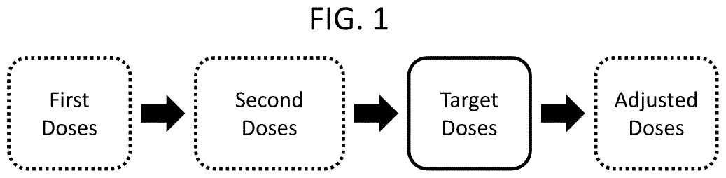

FIG. 1 shows a generalized method of treatment that lists each of the four steps. Whether the box is composed of a solid line or a broken line (i.e. a dashed line) indicates whether that step is performed, or whether it is optional, respectively. Since the box surrounding the text “target doses” is solid, this indicates that administering the target doses is performed. As discussed above, administering target doses involves administering both dC and dT, each independently within the range of 200 mg/kg/day to 600 mg/kg/day. For example, dC could be administered at 400 mg/kg/day and dT could also be administered at 400 mg/kg/day. In such a case, dC and dT would be administered at the same dosage. Alternatively, dC and dT could be administered at different dosages, such as 300 mg/kg/day of dC and 250 mg/kg/day of dT.

The box with the broken line on the right side of FIG. 1 recites “adjusted doses”, which refers to administering dC and dT in doses that are different from the doses of dC and dT administered during the target administrations. For example, as discussed below, a medical professional can monitor an indication of treatment efficacy, an indication of a side effect, or a combination thereof. Based on treatment efficacy, side effects, or a combination thereof, the dosages can be modified to be different from the dosages during the target administration. For example, if side effects were determined to be unacceptably severe, the adjusted doses can be lower than the target doses, e.g. in an attempt to maintain treatment efficacy while reducing side effects. Alternatively, the adjusted doses can be higher than the target doses, such as if side effects are low or absent and additional treatment efficacy is desired.

The boxes surrounding “first doses” and “second doses” in FIG. 1 have broken lines, indicating that those steps are optional. In some cases, both the first administration and the second administration are performed. In such cases, the dosages of dC and dT are incrementally increased from a baseline of zero dC and dT being administered to the target doses of dC and dT. In each case, the second doses are greater than the first doses, and the target doses are greater than the second doses. In some cases, the first administration is not performed, and only the second administration is performed. In other words, only one incremental dosage of dC and dT is investigated before administering the target doses to the subject. Performing the second administration and the optional first administration can be used to monitor side effects of the dC and dT, and to reduce the risk of causing severe side effects. In some cases, one or more of the initial doses is determined to provide sufficient treatment efficacy relative to any side effects, and therefore the initial doses can be maintained without increasing the dosages to the initially planned target doses.

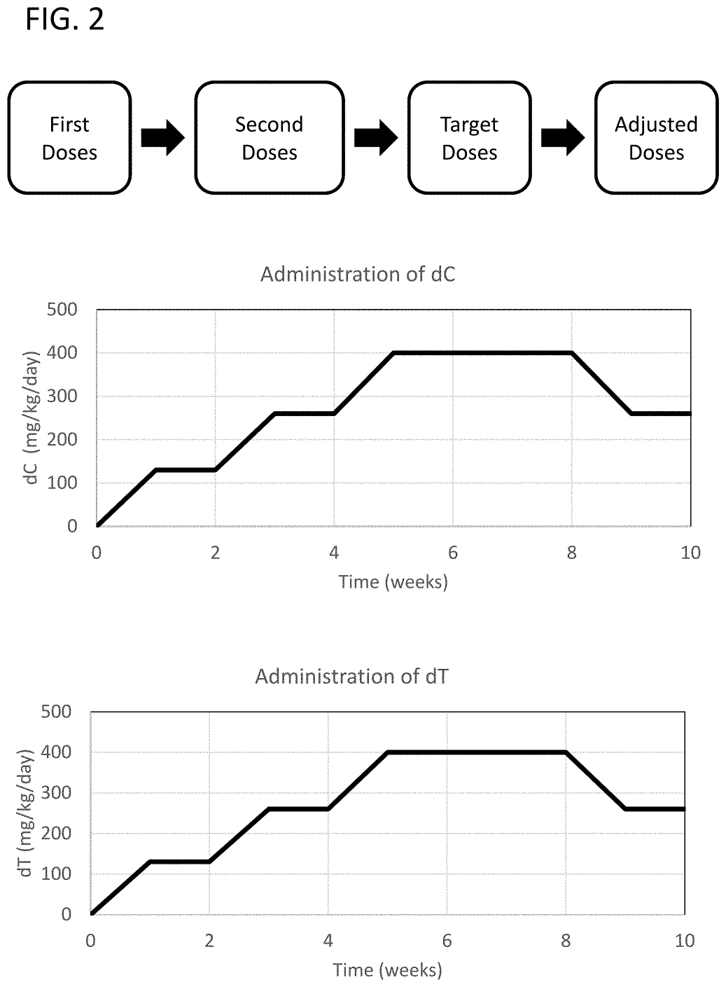

FIG. 2 shows an embodiment where all four of the steps are performed. At Week 0, which can be referred to as a baseline, the subject is not receiving dC or dT. Starting with Week 1, which corresponds to the first administrations, the subject is administered 130 mg/kg/day or dC and 130 mg/kg/day of dT. This dosage is continued to in Week 2. Optionally, the subject can be monitored for an indication of treatment efficacy, an indication of a side effect, or a combination thereof.

At Week 3, the doses of dC and dT are both increased to 260 mg/kg/day, corresponding to the second administration. This dosage is continued in Week 4. Optionally, the subject can be monitored for an indication of treatment efficacy, an indication of a side effect, or a combination thereof.

At Week 5, the doses of dC and dT are both increased to 400 mg/kg/day, corresponding to the target administration. This dosage is continued for Weeks 6, 7, and 8. Optionally, the subject can be monitored for an indication of treatment efficacy, an indication of a side effect, or a combination thereof.

At Week 9, the dosage of dC and dT are both decreased to 260 mg/kg/day. For example, the dosages might have been decreased because at the target dosages of 400 mg/kg/day treatment efficacy was determined to be sufficient, side effects were determined to be undesirably high, or a combination thereof. The dosage is continued in Week 10. Optionally, the subject can be monitored for an indication of treatment efficacy, an indication of a side effect, or a combination thereof. Although not shown in FIG. 2, the dosages can be further modified in the future any number of suitable times, such as 1 or more times, 2 or more times, 3 or more times, 4 or more times, or 5 or more times. Each further modification can independently be an increase in dosage or a decrease in dosage.

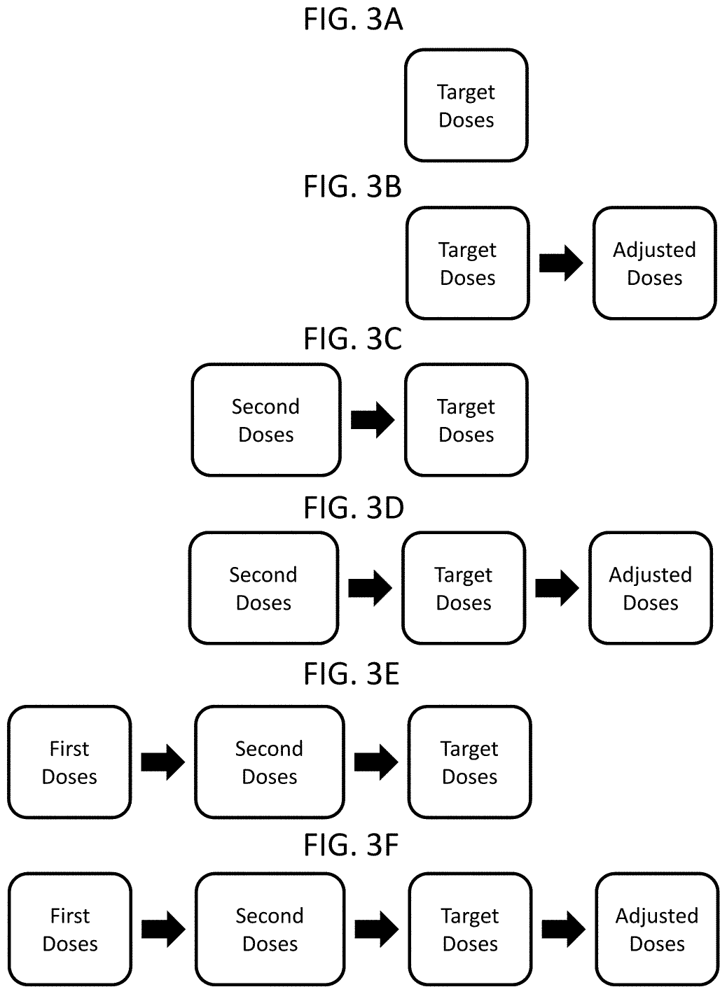

FIGS. 3A-3F show possible options for performing the generalized method of FIG. 1. The example of FIG. 2 is shown in FIG. 3F, which involves all four steps being performed. However, each of FIGS. 3A-3E can also be performed.

In FIGS. 3A-3B, the patient is administered the target doses without any previous trial dosages. In FIG. 3A, the target dose is continued without adjustment, whereas in FIG. 3B the dosage is adjusted one or more times.

In FIGS. 3C and 3D, the second administration is performed but not the first administration. In other words, the subject is administered one set of initial doses, such as to evaluate possible side effects, and then transitioned directly to the target doses. Optionally the target doses are further adjusted in the future.

In FIGS. 3E and 3F, both the first and second administrations are performed before the target administration. In other words, the subject is administered first doses, the subject is optionally monitored for treatment efficacy or side effects or both, and then the subject is transitioned to second doses that are higher than the first doses. The subject is optionally monitored at this stage, and then transitioned to the target doses. Optionally the dosages are adjusted after the target doses.

Although not shown in FIG. 1, any suitable number of initial doses can be administered to the subject before administering the target doses. For example, 1 or more, 2 or more, 3 or more, 4 or more, or 5 or more initial administrations can be performed. Although not shown in FIG. 1, any suitable number of adjusted doses can be administered to the subject after administering the target doses, such as 1 or more, 2 or more, 3 or more, 4 or more, or 5 or more. For example, immediately after the target administration a reduced dosage can be administered as the first adjusted administration, followed by a second adjusted dosage that is reduced compared to the first adjusted administration. In other words, upon each adjustment, the dosage can be increased or decreased. For example, the dosage can be increased for the first adjustment, increased for the second adjustment, and then decreased for the third adjustment. Any suitable combination of increases and decreases can be performed, for example: increase; decrease; increase then increase; increase then decrease; decrease then increase; decrease then decrease; increase then decrease then increase. After a suitable dosage has been determined, e.g. wherein treatment efficacy and side effects are determined to be appropriately balanced, these adjusted doses can be maintained, e.g. for 5 or more days, such as 25 or more, 50 or more, 100 or more, 150 or more, 200 or more, 250 or more, 300 or more, 400 or more, 600 or more, or 800 or more.

In some cases, the initial administrations are not performed, wherein the initial administration of dC and dT to the subject is at the target doses.

### Doses of Deoxycytidine and Deoxythymidine, and Duration of Administration

In some cases, the doses of dC and dT administered are equal. In some cases, the doses of dC and dT administered are unequal. In some of such cases, the dose of dC differs from the dose of dT by 10% or less. In other cases, the dose of dC differs from the dose of dT by more than 10%.

In some cases, the target doses of dC and dT are each independently between 200 mg/kg/day and 600 mg/kg/day, such as 250 to 550, 275 to 500, 325 to 475, 350 to 450, or 390 to 410. The target doses can be administered for 5 or more days, such as 25 or more, 50 or more, 100 or more, 150 or more, 200 or more, 250 or more, 300 or more, 400 or more, 600 or more, or 800 or more.

In some cases, the second doses are each 10 mg/kg/day or more. In some cases, the second doses are each independently 50 mg/kg/day or more and also at least 25 mg/kg/day less than the target dose. For example, if the target doses are 400 mg/kg/day, then the second doses can range from 50 mg/kg/day to 375 mg/kg/day. In some cases, the second doses are each independently between 100 mg/kg/day or more and also at least 50 mg/kg/day less than the target dose. In some cases, the second doses are each independently between 200 mg/kg/day and 300 mg/kg/day, such as 250 to 270. In some cases, the second doses are administered for between 5 days and 95 days, such as 5 days to 35 days.

In some cases, the first doses are each independently 10 mg/kg/day or more, such as 25 or more, 50 or more, or 70 or more. In some cases, the first doses are each independently 70 mg/kg/day or more and also at least 30 mg/kg/day less than the second doses. In some cases, the first doses each independently between 10 mg/kg/day and 250 mg/kg/day, such as between 50 and 200, between 90 and 180, or between 120 and 140. In some cases, the first doses are administered for between 5 days and 95 days, such as 5 days to 35 days.

In some cases, the adjusted doses are greater than the target doses, such as 105% or more of the target doses, 120% or more of the target doses, 140% or more of the target doses, or 160% or more of the target doses. In some cases, the adjusted doses are less than the target doses, such as 95% or less of the target doses, 90% or less of the target doses, 85% or less of the target doses, 80% or less of the target doses, 70% or less of the target doses, 60% or less of the target doses, or 40% or less of the target doses. In some cases, the adjusted doses are equal to the first doses or the second doses.

In some cases, the method further includes performing additional adjusted administrations. For example, target dosages of 400 mg/kg/day might have been determined to generate unacceptably severe side effects, and so the adjusted doses might be lower, such as at 260 mg/kg/day. Upon monitoring the patient, it might be observed that side effects have declined significantly but treatment efficacy has reduced, and thus a second adjusted dosage can be administered, e.g. between 260 and 400 mg/kg/day, such as 350 mg/kg/day. Any suitable number of adjusted doses can be administered to the subject after administering the target doses, such as 1 or more, 2 or more, 3 or more, 4 or more, or 5 or more.

### Monitoring the Subject

The subject can be monitored for an indication of treatment efficacy, an indication of a side effect, or both. The monitoring can be performed during the one or more administrations, such as during all of the administrations. The monitoring can also be performed for part of an administration, such as for the first 8 weeks of the target administration, but can optionally be discontinued. In some cases, adjusted doses are determined based on the monitoring. In some cases, the second dose is based on monitoring during the first administration. In some cases, the target doses are determined based on monitoring during the second administration.

In some cases, the indication of treatment efficacy is a motor function, such as: strength of a muscle, head control, rolling from a supine position to a side position, sitting in an upright position, transitioning from a sitting position to a standing position, standing, walking along a flat surface, walking up or down stairs, jumping, hopping, and running. In some cases, the indication of treatment efficacy is respiratory status, e.g. whether or not a ventilator is being used, end-tidal CO₂, or a result from a spirometry test. The indication of treatment efficacy can also be feeding tube usage, height, weight, body mass index (BMI), or head circumference. In some cases, the indication of treatment efficacy is a clinician-reported or self-reported quality of life. In some cases, the monitoring includes monitoring for two or more of the indications of treatment efficacy, such as 3 or more, 4 or more, or 5 or more. In some cases, the monitoring includes monitoring for one or more motor functions and one or more respiratory statuses.

In some cases, the monitoring includes monitoring for a possible side effect, such as diarrhea. In some case, the method includes administering a pharmaceutical composition for alleviating the side effect, such as an anti-diarrhea medication.

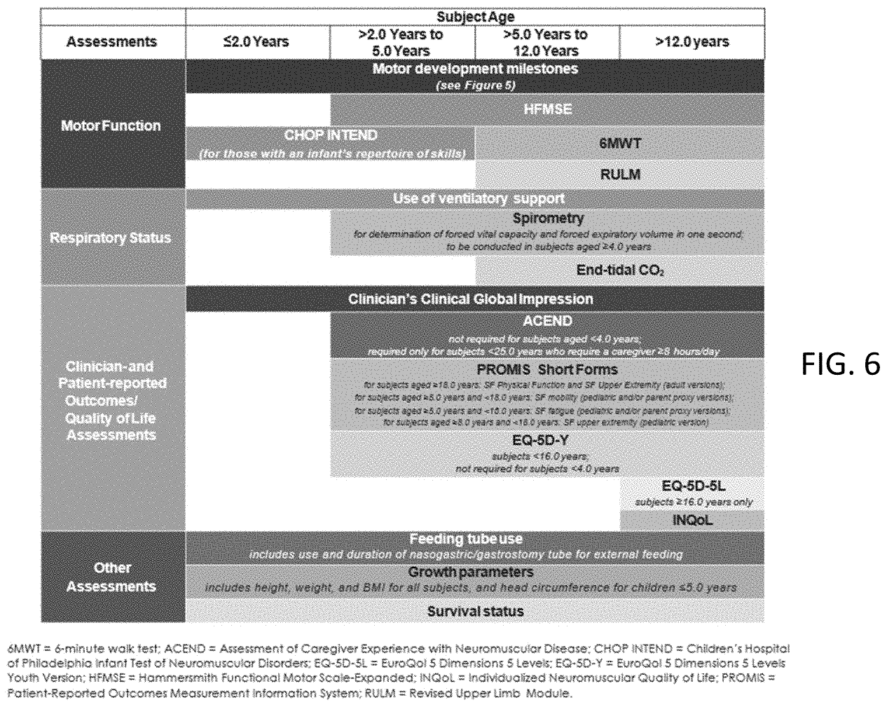

As shown in FIG. 6, various types and sub-types of monitoring can be performed, and sometimes these sub-types of monitoring are specific to certain age ranges. For instance, under the category of respiratory status, use of ventilator support can be monitored and an indication of treatment efficacy for all age ranges, e.g. less than 2 years, 2 to 5 years, 5 to 12 years, and greater than 12 years. In contrast, generally spirometry is only employed with subjects that are 2 years of age or greater.

Exemplary motor function tests include HFMSE, CHOP INTEND, 6MWT, and RULM. HFMSE refers to the Hammersmith Functional Motor Scale Expanded for SMA, and is usually employed for subjects 2 years or more of age. CHOP INTEND refers to the Children's Hospital of Philadelphia Infant Test of Neuromuscular Disorders, and is usually only used with subjects less than 5 years old. 6MWT refers to the Six-Minute Walk Test, which assesses spinal muscular atrophy. An example of the 6MWT is discussed in Montes et al. (Neurology, 2010, doi: 10.1212/WNL.0b013e3181d3e308). RULM refers to Revised Upper Limb Module for SMA, and is usually performed with subjects 5 years or older. As such, in some cases, monitoring motor function includes: performing a HFMSE test with a subject 2 years or older, performing a CHOP INTEND test with a subject 5 years or younger, performing a 6MWT test with a subject 5 years or older, performing a RULM test with a subject 5 years or older, or a combination thereof.

In some cases, monitoring respiratory status includes assessing use of ventilatory support at any age, performing a spirometry test with a subject of 2 years or older, performing an end-tidal CO₂ test with a subject 5 years or older, or a combination thereof. Sometimes, the spirometry is given to a subject 4 years or older. In some cases, the spirometry is assessed as the forced vital capacity and forced expiratory volume in one second.

Exemplary quality of life assessments include ACEND, PROMIS Short Forms, EQ-5D-Y, EQ-5D-5L, and INQoL. ACEND refers to Assessment of Caregiver Experience with Neuromuscular Disease. An exemplary reference describing ACEND is Matsumoto et al. (Journal of Pediatric Orthopaedics, 2011, doi:10.1097/BPO.0b013e31820fc522). In some cases the ACEND assessment is only performed on subjects 4 years or older. In some cases the ACEND assessment is only performed on subjects 4 to 25 years old who require a caregiver 8 or more hours per day. PROMIS refers to Patient-Reported Outcomes Measurement Information System. An exemplary reference describing PROMIS for assessing physical functioning in children at risk of neuromuscular illnesses is Rodday et al. (Journal of Patient-Reported Outcomes, 2017, doi: 0.1186/s41687-017-0011-8). Generically, EQ-5D is a standardized test for measuring generic health status. EQ refers to EuroQol and 5D refers to 5 dimensions. L refers to the number of levels. EQ-5D-Y refers to an assessment for children (Y; youth) that has 5 dimensions (5D) of mobility, looking after myself, doing usual activities, having pain or discomfort and feeling worried, sad or unhappy. An exemplary study using EQ-5D-Y is Kreimeir et al (Value in Health, 2019, doi: j.jval.2018.11.001). In some cases the EQ-5D-Y assessment is employed with subjects 4 to 16 years old. The EQ-5D-5L assessment includes the EQ-5D descriptive system and the EQ visual analogue scale (EQ VAS). EQ-5D-5L is another EQ-5D study. An exemplary EQ-5D-5L study is Lin et al (PLoS One, 2018, doi: 10.1371/journal.pone.0209344). In some cases the EQ-5D-5L is given to a subject ranging from 12 to 16 years old. INQoL refers to Individualized Neuromuscular Quality of Life questionnaires. An exemplary INQoL publication is Vincent et al (Neurology, 2007, doi:10.1212/01.wn1.0000257819.47628.41). In some cases the INQoL is given to a subject 12 years or older.

Other assessments include feeding tube use and growth parameters, e.g. height, weight, body mass index (BMI), and head circumference. In some cases, the monitoring includes assessing feeding tube use, height, weight, BMI, or a combination thereof for patients of any age. In some cases, the monitoring includes assessing head circumference for subjects aged 5 years or younger.

### Route of Administration and Subject

In some cases, the dC and dT are administered as part of a single pharmaceutical composition. For example, the subject can be orally administered one or more pills that each include a mixture of dC and dT. Alternatively, the dC and dT are administered as separate pharmaceutical compositions. For example, the subject can be orally administered one or more pills that each include dC but no dT, and also orally administered one or more pills that each include dT but no dC.

The dC and dT can be administered by any suitable route, such as orally, intrathecally, enterally, intravenously, or a combination thereof. In some cases, the dC and dT are administered orally.

The administrations can be performed once a day, twice a day, three times a day, four times a day, five times a day, or six or more times a day. In some cases, the administrations are performed three times a day. In some cases, each administrations over a day involve administering an equal dose of dT, wherein each administrations over a day involve administering an equal dose of dC. For example, if a subject is administered 400 mg/kg/day of dC and the administrations are performed twice a day, then the subject is administered 200 mg/kg at each of the two daily administrations. In some cases, the subject has infantile-onset myopathy, i.e. the onset of symptoms of TK2 deficiency was from birth to 2 years of age. In some cases, the subject has childhood-onset myopathy, i.e. the onset of symptoms of TK2 deficiency was between 3 years and 12 years of age. In some cases, the subject has late on-set myopathy, i.e. the onset of symptoms of TK2 deficiency was after age 12.

In some cases, the human subject is 0 through 2 years of age. In some cases, the human subject is from 3 through 12 years of age. In some cases, the human subject is greater than 12 years of age.

Notwithstanding the appended claims, the disclosure is also defined by the following clauses:
1. A method of treating thymidine kinase 2 (TK2) deficiency in a human subject with deoxycytidine (dC) and deoxythymidine (dT), comprising:
   optionally administering first doses of dC and dT to the subject;
   optionally administering second doses of dC and dT to the subject, wherein the second doses are greater than the first doses;
   administering target doses of dC and dT to the subject, wherein the target doses are each independently between 200 mg/kg/day and 600 mg/kg/day and also greater than the second doses; and
   optionally administering adjusted doses of dC and dT to the subject, wherein each of the adjusted doses are different than the target doses.
2. The method of clause 1, wherein the target doses of dC and dT are each independently between 275 mg/kg/day and 500 mg/kg/day.
3. The method of clause 2, wherein the target doses of dC and dT are each independently between 325 mg/kg/day and 475 mg/kg/day.
4. The method of clause 3, wherein the target doses of dC and dT are each independently between 390 mg/kg/day and 410 mg/kg/day.
5. The method of any one of clauses 1-4, wherein the target doses are administered for 25 days or more.
6. The method of clause 5, wherein the target doses are administered for 100 days or more.
7. The method of clause 6, wherein the target doses are administered for 250 days or more.
8. The method of any one of clauses 1-7, wherein administering the second doses is performed.
9. The method of clause 8, wherein the second doses are each independently 100 mg/kg/day or more and also at least 50 mg/kg/day less than the target doses.
10. The method of clause 9, wherein the second doses are each independently between 200 mg/kg/day and 300 mg/kg/day.
11. The method of clause 10, wherein second doses are each independently between 250 mg/kg/day and 270 mg/kg/day.
12. The method of any one of clauses 8-11, wherein the second doses are administered for between 5 days and 95 days.
13. The method of clause 12, wherein the second doses are administered for between 5 days and 35 days.
14. The method of any one of clauses 8-13, wherein administering the first doses is performed.
15. The method of clause 14, wherein the first doses are each independently 70 mg/kg/day or more and also at least 30 mg/kg/day less than the second doses.
16. The method of clause 15, wherein the first doses are each independently between 90 mg/kg/day and 180 mg/kg/day.
17. The method of clause 16, wherein first doses are each independently between 120 mg/kg/day and 140 mg/kg/day.
18. The method of any one of clauses 14-17, wherein the first doses are administered for between 5 days and 95 days.
19. The method of clause 18, wherein the first doses are administered for between 5 days and 35 days.
20. The method of any one of clauses 1-19, wherein administering the adjusted doses is performed.
21. The method of clause 20, wherein the adjusted doses are greater than the target doses.
22. The method of clause 21, wherein the adjusted doses are 120% or more of the target doses.
23. The method of clause 20, wherein the adjusted doses are less than the target doses.
24. The method of clause 23, wherein the adjusted doses are 80% or less of the target doses.
25. The method of clause 23 or 24, wherein the adjusted doses are equal to the first doses or the second doses.
26. The method of any one of clauses 1-25, further comprising monitoring for an indication of treatment efficacy, a possible side effect of the administration, or a combination thereof during one or more of the administering steps.
27. The method of clause 26, further comprising monitoring both for an indication of treatment efficacy and a possible side effect of the administration.
28. The method of clause 26 or 27, wherein the monitoring is performed during all of the administering steps.
29. The method of any one of clauses 26-28, wherein the adjusted doses are based on the monitoring.
30. The method of any one of clauses 26-29, further comprising monitoring both for an indication of treatment efficacy and a possible side effect of the administration during all of the administering steps, wherein the adjusted doses are based on the monitoring.
31. The method of any one of clauses 26-30, wherein the indication of treatment efficacy is a motor function.
32. The method of clause 31, wherein the motor function is strength of a muscle.
33. The method of clause 32, wherein the motor function is selected from the group consisting of: head control, rolling from a supine position to a side position, sitting in an upright position, transitioning from a sitting position to a standing position, standing, walking along a flat surface, walking up or down stairs, jumping, hopping, and running.
34. The method of any one of clauses 26-30, wherein the indication of treatment efficacy is a respiratory status.
35. The method of clause 34, wherein the respiratory status is whether or not a ventilator is being used, end-tidal CO₂, or a result from a spirometry test.
36. The method of any one of clauses 26-30, wherein the indication of treatment efficacy is feeding tube usage.
37. The method of any one of clauses 26-30, wherein the indication of treatment efficacy is height, weight, body mass index (BMI), or head circumference.
38. The method of any one of clauses 26-30, wherein the indication of treatment efficacy is a clinician-reported or self-reported quality of life.
39. The method of any one of clauses 26-38, further comprising monitoring a possible side effect.
40. The method of clause 39, wherein the side effect is diarrhea.
41. The method of clause 39 or 41, further comprising administering a pharmaceutical composition for alleviating the side effect.
42. The method of clause 31, wherein the pharmaceutical composition for alleviating the side effect is a diarrhea medication.
43. The method of any one of clauses 1-42, wherein the second doses are administered and the adjusted doses are administered.
44. The method of any one of clauses 1-42, wherein the first doses are administered, the second doses are administered, and the adjusted doses are administered.
45. The method of any one of clauses 1-44, wherein the dC and dT are administered as part of a single pharmaceutical composition.
46. The method of any one of clauses 1-44, wherein the dC and dT are administered as parts of separate pharmaceutical compositions.
47. The method of any one of clauses 1-46, wherein the dC and dT are administered orally, intrathecally, enterally, intravenously, or a combination thereof.
48. The method of clause 47, wherein the dC and dT are administered orally.
49. The method of any one of clauses 1-48, wherein each administration is performed once a day, twice a day, three times a day, four times a day, or five times a day.
50. The method of clause 49, wherein each administration is performed three times a day.
51. The method of clause 49 or 50, wherein each administrations over a day involve administering an equal dose of dT, wherein each administrations over a day involve administering an equal dose of dC.
52. The method of clause 49 or 50, wherein the dose of dC differs from the dose of dT by 10% or less in the target administrations.
53. The method of clause 52, wherein the dose of dC is equal to the dose of dT in the target administrations.
54. The method of any one of clauses 1-53, wherein the human subject is 0 through 2 years of age.
55. The method of any one of clauses 1-53, wherein the human subject is from 3 through 12 years of age.
56. The method of any one of clauses 1-53, wherein the human subject is greater than 12 years of age.

### EXAMPLE

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. doses, temperature, etc.) but some experimental errors and deviations should be accounted for.

All subject were genetically confirmed to have TK2 deficiency. All subjects lacked other confirmed genetic or polygenic diseases that might confound evaluating the efficacy of administering dT and dC. All subjects had normal liver function test results. Subjects that had been treated within the preceding one year with nucleotide or nucleoside therapy were excluded from the study. Subjects with EtCO₂ of greater than 45 mmHg were excluded if not on ventilator support.

The study included 25 subjects. Group A included 10 subjects that had significant loss or decline (regression) in previously achieved motor milestones or developmental delay in achieving motor milestones. Group B included 15 subjects that were unable to perform at least one of the motor milestones.

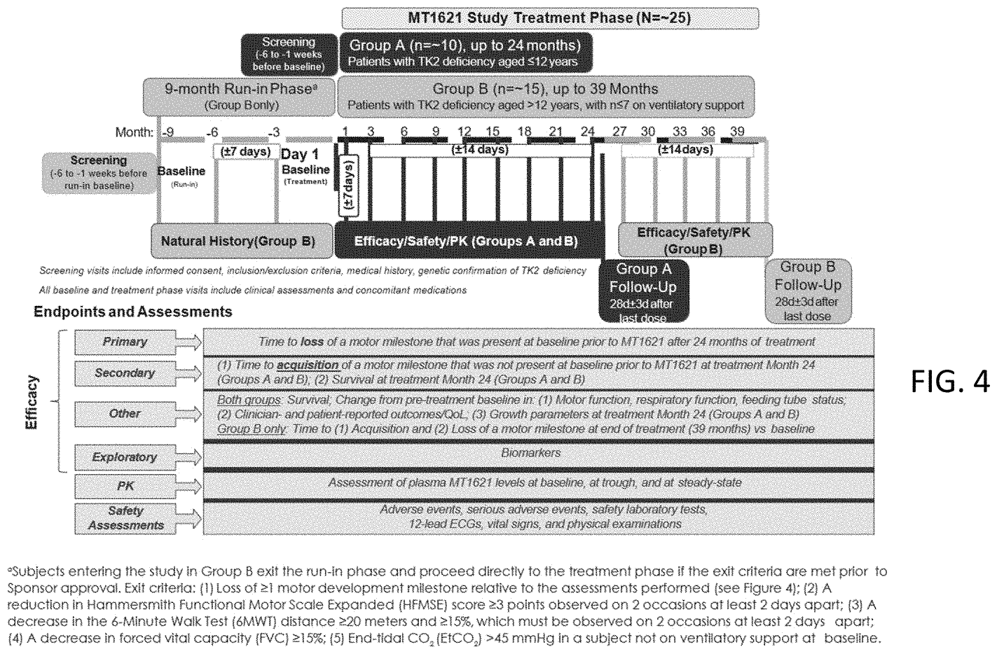

FIG. 4 shows an overview of the study design wherein the 25 subjects were administered dT and dC. The subjects were administered “MT1621”, which is a pharmaceutical composition including equal doses of dC and dT.

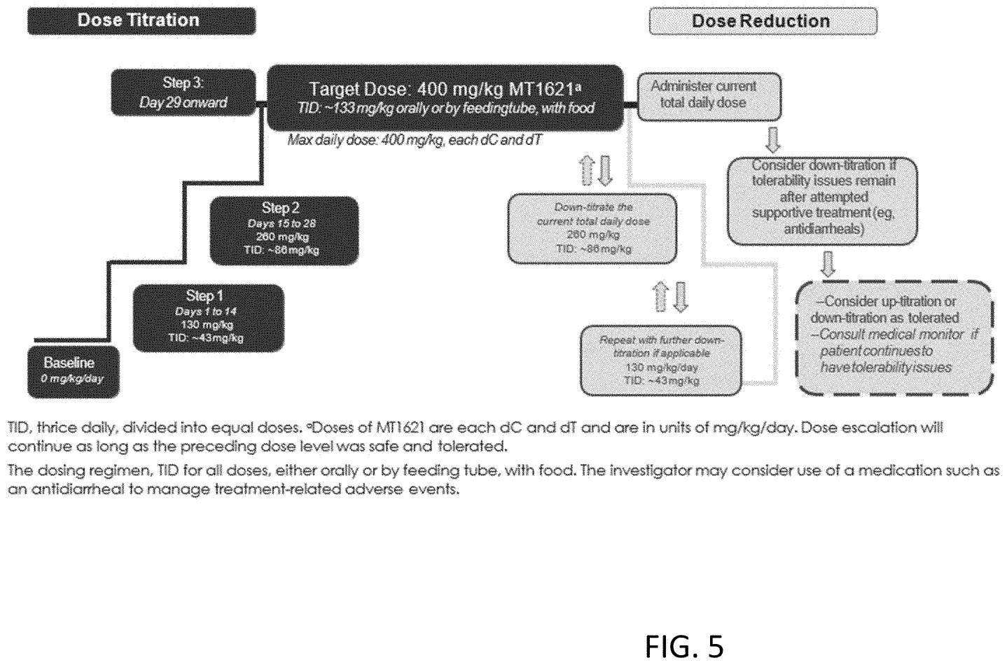

FIG. 5 shows dose titration and reduction schedules for the study design of FIG. 4. At baseline, subjects received no dC or dT. During Step 1, which corresponds to the first administration described herein, subjects were administered ˜43 mg/kg three times a day (TID) for a total daily dose of 130 mg/kg. During Step 2, which corresponds to the second administration, subjects where administered 260 mg/kg/day in three equal doses per day. During Step 3, which corresponds to the target administrations, subjects were administered 400 mg/kg/day. In the dose reduction stage, the dosages were optionally reduced (i.e. down-titrated) if side effects (i.e. tolerability issues) remained after supportive treatments. Optionally the doses were then up-titrated (i.e. increased) based on a balance between treatment efficacy and side effects.

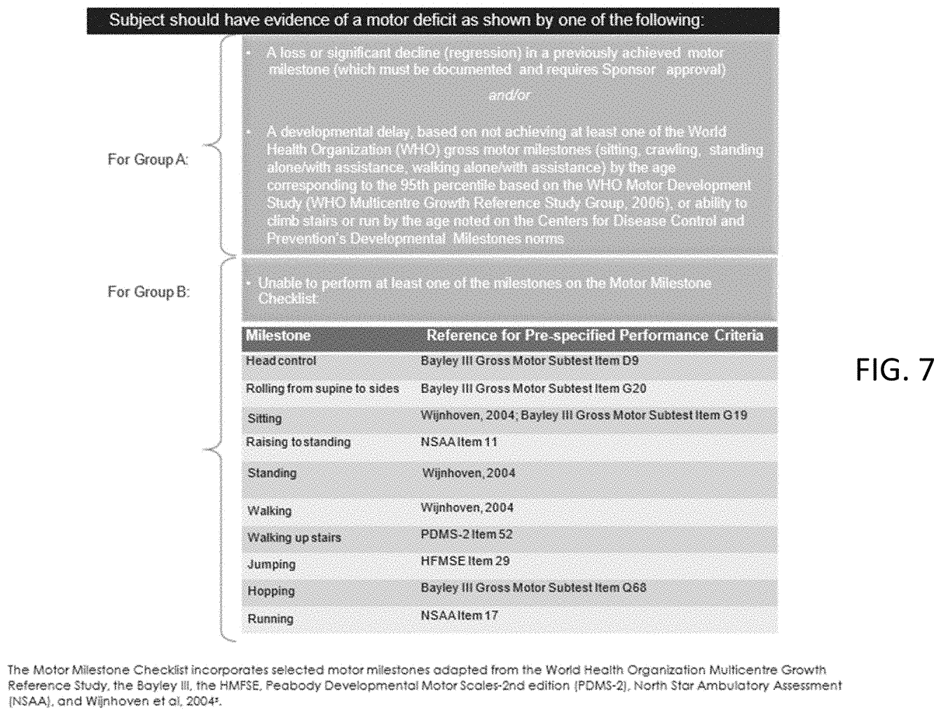

FIG. 6 shows the indications of treatment efficacy that were used to monitor each subject depending on the age of the subject. The categories of FIG. 6 include motor function, respiratory status, clinician- and patient-reported outcomes and quality of life assessments, and other assessments (i.e. feeding tube use, growth parameters such as height and weight, and survival or death). FIG. 7 shows a detailed discussion of the types of motor function assessments employed during the monitoring. The categories of FIG. 7 for Group A include loss or decline in a previous motor function and a delay in motor development according to the World Health Organization's WHO Multicentre Growth Reference Study Group of 2006. Group B categories in FIG. 7 include head control, rolling from supine to sides, sitting, raising to standing, standing, walking, walking up stairs, jumping, hopping, and running.

As such, each of the subjects were administered MT1621, which is a pharmaceutical composition including equal doses of dT and dC, for the duration of the study period. Each subject was also monitored for the indications of treatment efficacy discussed in FIGS. 6 and 7 throughout the study period.

Statistical Methods:

Data was summarized using descriptive statistics. Continuous data: sample size, mean, standard deviation, median, ranges, 25th and 75th percentiles. Categorical data: number, percentage of subjects

Time-to-event methods (Cox models and Kaplan-Meier plots) and longitudinal data analysis methods (mixed model analysis of variance; general estimating equations) will be used to evaluate relationships between potential prognostic variables and outcome measures. Test-based 95% confidence intervals will exclude risk profiles for loss of motor milestones and acquisition of motor milestones. All inferential statistics were focused on point and interval estimation of key parameters

Primary and secondary efficacy endpoints were analyzed using the combined data from Group A and Group B, with group as a stratum variable for analyses. Subgroup analyses were performed on individual groups.

Sample size: Power analyses determined that a sample size of 25 subjects is sufficient to create a test-based 95% confidence interval that does not cover the hazard ratio of one if the true hazard ratio is: <0.3 for the primary efficacy endpoint and >2 for the secondary efficacy endpoint.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(1) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A method of treating thymidine kinase 2 (TK2) deficiency in a human subject, comprising:
administering to the subject a therapeutically effective amount of target doses of deoxycytidine (dC) and deoxythymidine (dT) wherein the target doses are each independently between 200 mg/kg/day and 600 mg/kg/day.

2. The method of claim 1, wherein the target doses of dC and dT are each independently between 390 mg/kg/day and 410 mg/kg/day and are administered for 250 days or more.

3. The method of claim 2, further comprising:
administering second doses wherein the second doses are each independently between 250 mg/kg/day and 270 mg/kg/day and at least 50 mg/kg/day less than the target doses, and are administered for between 5 days and 35 days.

4. The method of claim 3, further comprising:
administering first doses which are each independently 70 mg/kg/day or more and at least 30 mg/kg/day less than the second doses.

5. The method of claim 4, wherein first doses are each independently between 120 mg/kg/day and 140 mg/kg/day and are administered for between 5 days and 35 days.

6. The method of claim 5, further comprising:
administering adjusted doses.

7. The method of claim 6, wherein the adjusted doses are greater than the target doses.

8. The method of claim 6, wherein the adjusted doses are 120% or more of the target doses.

9. The method of claim 6, wherein the adjusted doses are less than the target doses.

10. The method of claim 6, wherein the adjusted doses are 80% or less of the target doses.

11. The method of claim 6, further comprising:
monitoring for an indication of treatment efficacy, a possible side effect of the administration or a combination thereof during one or more of the administering steps.

12. The method of claim 6, further comprising:
monitoring both for an indication of treatment efficacy and a possible side effect of the administration.

13. The method of claim 12, wherein the monitoring is performed during all the administering steps.

14. The method of claim 13, wherein the adjusted doses are based on the monitoring.

15. The method of claim 14, further comprising:
monitoring both for an indication of treatment efficacy and a possible side effect of the administration during all the administering steps, wherein the adjusted doses are based on the monitoring.

16. The method of claim 15, wherein the indication of treatment efficacy is a motor function.

17. The method of claim 15, wherein the motor function is strength of a muscle.

18. The method of claim 17, wherein the motor function is selected from the group consisting of: head control, rolling from a supine position to a side position, sitting in an upright position, transitioning from a sitting position to a standing position, standing, walking along a flat surface, walking up or down stairs, jumping, hopping, and running.

19. The method of claim 1, wherein the dC and dT are administered as parts of separate pharmaceutical compositions.

20. The method of claim 1, wherein the dC and dT are administered orally, intrathecally, enterally, intravenously, or a combination thereof.
